(19) United States
(12) Patent Application Publication
(10) Pub. No.: US 2019/0231352 A1
(43) Pub. Date: Aug. 1, 2019

(54) MEDICAL CLIP

(71) Applicant: KANEKA CORPORATION, Osaka-shi, Osaka (JP)

(72) Inventor: Naotake MAEKUBO, Okaya-shi (JP)

(73) Assignee: KANEKA CORPORATION, Osaka-shi, Osaka (JP)

(21) Appl. No.: 15/769,823

(22) PCT Filed: Oct. 4, 2016
(86) PCT No.: PCT/JP2016/079515

§ 371 Date: Apr. 20, 2018

(30) Foreign Application Priority Data
Oct. 23, 2015 (JP) 2015-209355

(51) Int. Cl. A61B 17/122

(52) U.S. Cl. CPC A61B 17/122; A61B 1/00087

(57) ABSTRACT

An object of the present invention is to provide a medical clip that enables the adjustment of a holding position or a closing angle until the very moment of closing the clip. A medical clip (1) comprises: a ring-shaped tightening member (20); and a clip body (10) including two holding members (11 and 12), wherein each of the two holding members (11 and 12) has a base end part (A), a curved part (B) that is bent inward in a radial direction of the tightening member (20), a wide part (C) that is wider than a minimum width of the curved part (B) and a holding part (D) for holding a target site in this order from a proximal side thereof, and the two holding members (11 and 12) are located so as to face each other and joined to each other at the base end part (A); wherein at least a part of the clip body (10) is enclosed by the tightening member (20), and a bending part (E) that is bent outward in the radial direction of the tightening member (20) is formed at a section of the wide part (C) of the holding member (11 and 12).

### TECHNICAL FIELD

The present invention relates to a medical clip for holding a target site mainly in a treatment with an endoscope.

### BACKGROUND ART

Conventionally, endoscopic submucosal dissection (ESD) and endoscopic mucosal resection (EMR) are adopted for early-stage cancer surgeries with an endoscope on organs such as esophagi or stomachs, which have only a small space for conducting a surgery. In ESD and EMR, some physiological saline solution or hyaluronic acid is infused between the muscular layer and the submucosal layer at a lesion to raise the lesion and surely excise the layers at the lesion. The lesion is held with an auxiliary clip with a line member and exfoliated by pulling the line member to the proximal side, and excised at the bottom with a surgical knife such as an electric scalpel to be removed from the body.

For example, Patent Document 1 discloses a clip for an endoscope that includes a clip member made by bending an elastic plate, and a ring-shaped clip tightening ring. The clip member includes rear parallel parts extending straight from the rear end part (the proximal side) toward the front end part (the distal side) of the clip member in parallel to each other in the rear part of the clip member in the front and rear direction, intermediate arc parts spreading outward in an arc manner from the front ends of the rear parallel parts, and front arm parts extending forward from the front ends of the intermediate arc parts. The intermediate arc parts of the clip member have the same width as the rear parallel parts. The front arm parts have the same width as the intermediate arc parts only in small areas near the rear ends adjacent to the boundary with the intermediate arc parts, and next to the small areas are tightening ring engaging parts, on which the tightening ring is firmly fitted. The tightening ring engaging parts have a larger width at the front end parts with which the front end of the clip tightening ring comes into contact, and thus function as a stopper to prevent the tightening ring from moving forward.

### PRIOR ART DOCUMENTS

### Citation List

### PATENT DOCUMENTS

### Patent Document 1

Japanese Unexamined Patent Application Publication No. 2009-11784

### SUMMARY OF THE INVENTION

### Problems to be Solved by the Invention

Once the tightening ring moves from the rear parallel parts toward the distal side and go over the boundary between the intermediate arc parts, which are formed by bending, and the front arm parts, the clip for an endoscope of Patent Document 1 is closed and cannot be opened anymore. Even if the clip is closed at a wrong holding position or at a wrong closing angle, the clip cannot be opened again because the tightening ring cannot be moved back in the proximal direction. This means that it is very difficult to correct the wrong holding operation.

An object of the present invention is to provide a medical clip that enables the adjustment of a holding position or a closing angle until the very moment of closing the clip.

### Solution to the Problems

A medical clip of the present invention which is able to achieve the above object comprises: a ring-shaped tightening member; and a clip body including two holding members, wherein each of the two holding members has a base end part, a curved part that is bent inward in a radial direction of the tightening member, a wide part that is wider than a minimum width of the curved part and a holding part for holding a target site in this order from a proximal side thereof, and the two holding members are located so as to face each other and joined to each other at the base end part; wherein at least a part of the clip body is enclosed by the tightening member, and a bending part that is bent outward in the radial direction of the tightening member is formed at a section of the wide part of the holding member. In the present invention, the bending parts of the holding members are bent outward in the radial direction of the tightening member. When the tightening member, which moves from the proximal side to the distal side, goes over the bending parts, the tightening member comes into contact with the holding members. At this time, the inner surface of the tightening member applies a pressing force to the holding members in the direction for closing the clip. In the present invention, since the tightening member tends to come into contact with the wide parts of the holding members in the direction perpendicular to the opening-and-closing direction of the clip, the wide parts temporarily stop the tightening member on the holding members. In the medical clip of the present invention, as the tightening member moves in an area proximal to the bending parts of the wide parts of the holding members in the longitudinal direction of the clip body, the tightening member gradually closes the clip in cooperation with the wide parts to adjust a holding position and a closing angle.

In the medical clip of the present invention, the wide part is preferably narrower than a maximum width of the holding part. This prevents the tightening member, which has gone over the bending parts of the holding members toward the distal sides of the holding parts, from dropping off from the clip body.

A narrow part that is narrower than a width of the wide part is preferably formed between the holding part and the wide part in the holding member. This shortens the section to be in contact with the tightening member in the longitudinal direction of the clip body after the distal end of the tightening member goes over the wide parts with the bending parts to come close to the holding parts, which enables the holding of a target site with a smaller power.

An inner diameter of the tightening member is preferably shorter than a maximum width of the holding part. After going over the bending parts of the holding members in the distal direction, the tightening member comes into contact with at least the areas having the maximum width of the holding parts. This prevents the tightening member from moving to the distal sides of the holding parts and dropping off from the clip body.

The wide part has a distal section, a proximal section and a central section between the distal section and the proximal section, that are formed by equally dividing the wide part into three sections in a longitudinal direction of the clip body, and the bending part is preferably formed at the distal section or the central section of the wide part. This lengthens the section where the clip is gradually closed while adjusting a holding position and a closing angle.

To enhance a rigidity of the holding part of the holding member, at least a part of the holding part is preferably formed in an arc shape that is bent outward in the radial direction of the tightening member in a cross-section perpendicular to a longitudinal direction of the holding part.

A claw part is preferably formed at a distal end of the holding part. The claw parts cut into a target site when the holding parts hold the target site, which ensures the holding of the target site.

A curved-surface is preferably formed at the distal end of the holding part. The holding parts having the curves at the distal ends enable the claw parts to come into contact with a target site in the direction perpendicular to the longitudinal direction, which enables the distal ends of the holding parts or the claw parts to easily cut into the target site.

The two holding members are preferably in contact with each other at a proximal end part of the curved part. Compared to the clip having the holding members separated from each other at the proximal end parts of the curved parts, the clip of this type can spread wide at the distal ends of the holding parts, which facilitates the holding of a target site not only at the distal sides of the holding parts but also in the entire areas of the holding parts.

The two holding members are preferably integrally formed at the base end part. Such a medical clip is suitably used for a clip device in which the line member for longitudinally moving the clip body is directly connected with the base end parts of the holding members.

The two holding members are preferably unjoined to each other at a proximal end of the base end part. Such a medical clip is suitably used for a clip device in which the line member for longitudinally moving the clip body is indirectly connected with the clip body via a joining member to engage with the proximal end parts of the base end parts of the clip body.

### Effects of the Invention

In the medical clip of the present invention, as the tightening member moves in an area proximal to the bending parts of the wide parts of the holding members in the longitudinal direction of the clip body, the tightening member gradually closes the clip in cooperation with the wide parts to adjust a holding position and a closing angle. The medical clip of the present invention thus prevents waste of clips due to misoperation of the clips in a holding operation and shortens surgical time.

### BRIEF DESCRIPTION OF THE DRAWINGS

FIG. 1 is a perspective view of a medical clip of the present invention.

FIG. 2 is a side view of the medical clip of the present invention.

FIG. 3 is a plan view of a clip body of the present invention.

FIG. 4 is a side view illustrating the clip body according to another embodiment of the present invention.

FIG. 5 is a side view of the clip body of the present invention.

FIG. 6 is a rear view illustrating the clip body according to still another embodiment of the present invention.

FIG. 7 is a side view illustrating the clip body according to still another embodiment of the present invention.

### MODE FOR CARRYING OUT THE INVENTION

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

In the present invention, a medical clip is an instrument for holding a target site such as a lesion in an organ in an endoscopic operation for sealing, countertraction, stanching, suturing, or marking. In this specification, a medical clip may simply be called “a clip.”

In the present invention, a longitudinal direction refers to a longitudinal direction of a clip body, and a proximal side in the longitudinal direction refers to a direction of an operator's hand side, while a distal side refers to a direction opposite to the proximal side. In the present invention, a radial direction refers to a radial direction of a tightening member, and an inner side in the radial direction refers to a direction toward a center of the tightening member, while an outer side refers to a radiation direction of the tightening member. A width of a holding member of the clip body refers to a length of the holding member in a direction perpendicular to a longitudinal direction in a plan view of the clip body.

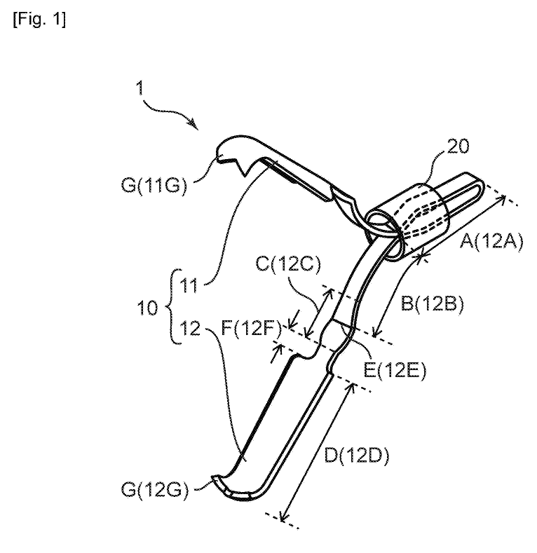

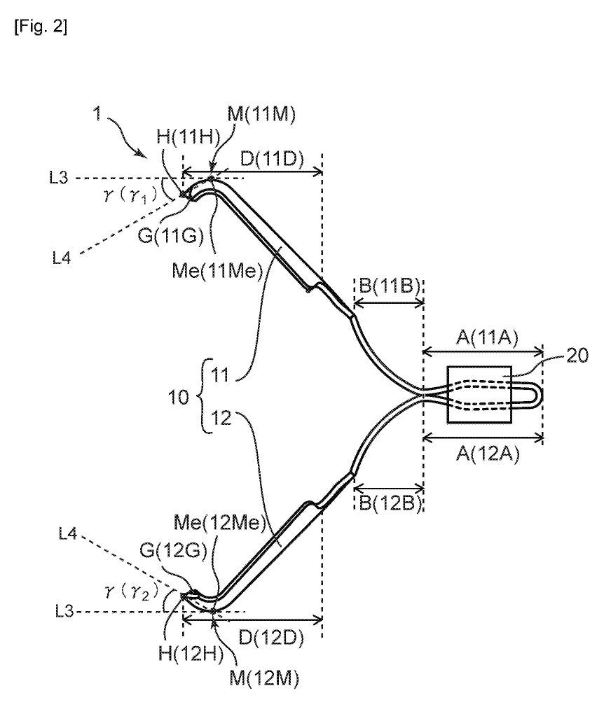

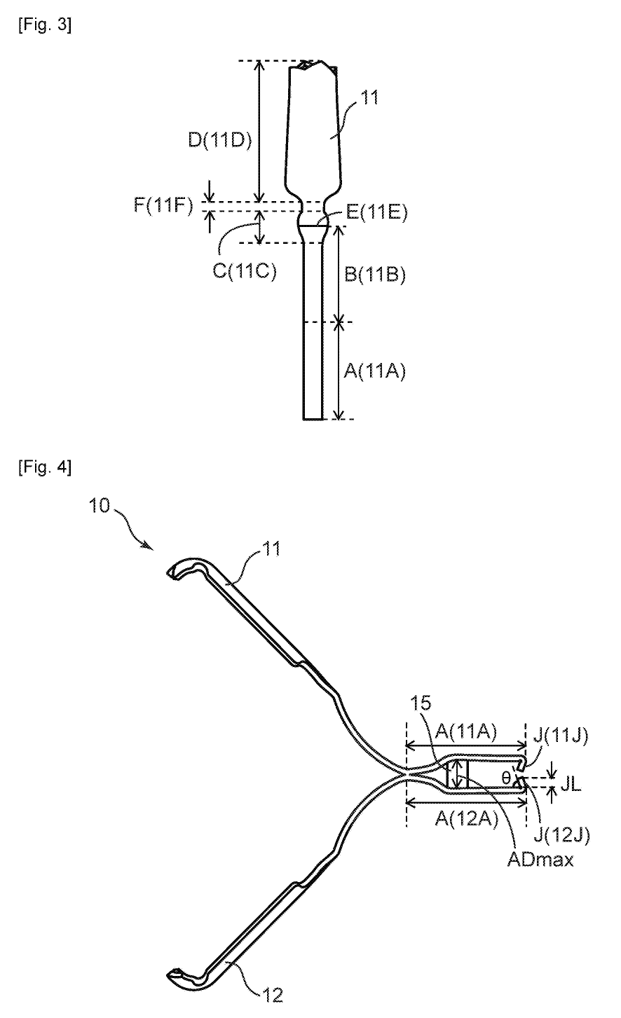

FIG. 1 is a perspective view of a medical clip of the present invention, FIG. 2 is a side view of the medical clip of the present invention, and FIG. 3 is a plan view of a clip body of the present invention. A medical clip 1 of the present invention comprises a clip body 10 and a tightening member 20.

In the clip body 10, the two holding members 11 and 12 are located so as to face each other. At least a part of the clip body 10 is enclosed by the tightening member 20, and the tightening member 20 is formed ring-shaped.

The clip 10 is closed in the following way. First, the tightening member 20 is placed around the proximal-side outer part of the clip body 10 in an open state. Then, the tightening member 20 is moved to the distal side of the clip body 10. As the tightening member 20 moves toward the distal side, the tightening member 20 applies an inward pressure to the holding members 11 and 12 in the radial direction, so that the holding members 11 and 12 come close to each other until the clip 1 closes. A configuration of the tightening member 20 and the clip body 10 is specifically described in the following.

At least a part of the clip body 10 is enclosed by the tightening member 20, and the tightening member 20 is formed ring-shaped. Specifically, the tightening member 20 is located at an outer side of the clip body 10 so as to be movable in the longitudinal direction of the clip body 10.

In the longitudinal direction of the clip body 10, a length of the tightening member 20 is preferably 4% or more and 30% or less of the clip body 10 in a closed state. Specifically, a length of the clip body 10 in the closed state is about 12 mm, for example. Accordingly, the length in the longitudinal direction of the tightening member 20 is preferably 0.3 mm or more, more preferably 1 mm or more, however, preferably 4 mm or less, and more preferably 3 mm or less.

The tightening member 20 may be a single ring-shaped member (shown in FIG. 1) or a ring-shaped member (not shown) formed of a wound wire rod in the form of a coil. The shape of the tightening member 20 is a circular ring shape or a polygonal ring shape, for example. The tightening member 20 may be closed in the circumferential direction, or open in the circumferential direction to have a C-shaped cross section perpendicular to the longitudinal direction, for example.

The clip 1 of the present invention is preferably used for a clip device (not shown) that is inserted into a forceps channel of an endoscope, and performs an operation of holding the target site such as a lesion by controlling the opening and closing of clip 1. For example, the clip device comprises an outer tubular body, an inner tubular body provided in the outer tubular body, and a line member provided in the inner tubular body. The clip 1 is located at a distal side of the inner tubular body and connected to the line member. The outer diameter of the tightening member 20 is preferably larger than the inner diameter of the inner tubular body of the clip device. This enables the proximal end of the tightening member 20 to come into contact with the distal end of the inner tubular body when the line member is pulled in the proximal direction, so that only the clip body 10 is pulled into the inner tubular body. As a result, the tightening member 20 moves to the distal side of the clip body 10 to close the clip.

The tightening member 20 may be composed of a metal material or elastic material. The tightening member 20 preferably has a biocompatibility. The tightening member 20 is preferably composed of a Ni—Ti alloy or a stainless steel such as SUS304, SUS631, for example.

The clip body 10 is a member for holding a target site such as a lesion. The two holding members 11 and 12 are located so as to face each other, and at least a part of the clip body 10 is enclosed by the tightening member 20. Specifically, in the clip body 10, one holding member 11 and the other holding member 12 are located so as to face each other.

Each of the two holding members 11 and 12 has a base end part A, a curved part B that is bent inward in a radial direction of the tightening member 20, a wide part C that is wider than a minimum width of the curved part B and a holding part D for holding a target site in this order from a proximal side thereof. Specifically, the one holding member 11 has a base end part 11A, a curved part 11B, a wide part 11C and a holding part 11D in this order from a proximal side thereof. The other holding member 12 has a base end part 12A, a curved part 12B, a wide part 12C and a holding part 12D in this order from a proximal side thereof as well as the one holding member 11. The two holding members 11 and 12 are joined to each other at the base end parts 11A and 12A.

The holding members 11 and 12 are formed of a band-shaped member having a thickness of 0.1 mm or more and 0.5 mm or less, for example. The holding members 11 and 12 may be formed by bending the band-shaped member or by preparing individual band-shaped members for the base end parts A, the curved parts B, and the holding parts D and joining the members by laser welding, for example.

As shown in FIG. 2, the holding members 11 and 12 may symmetrically be formed with respect to the longitudinal axis of the clip body 10. This enables the holding members 11 and 12 to timely come close to each other, which facilitates the holding of a target site.

As shown in FIG. 2, the holding members 11 and 12 may have substantially the same length in the longitudinal direction. In this case, the two holding members 11 and 12 come close to each other mainly at the distal ends of the holding parts D.

The one holding member 11 may be longer than the other holding member 12 in the longitudinal direction (not shown). In this case, the two holding members 11 and 12 come close to each other not only at the distal ends of the holding parts D but also at positions proximal to the distal ends of the holding parts D, which enables the holding of a target site in all the longitudinal area of the clip body 10.

The clip body 10 including the holding members 11 and 12 preferably has an excellent strength and biocompatibility. The clip body 10 is composed of a Ni—Ti alloy or a stainless steel such as SUS304, SUS631, for example. However, the holding members 11 and 12 may be composed of the same material or the different material.

The size of the clip 1 is not limited, however, for the purpose of withdrawing the clip 1 through the forceps channel of an endoscope or together with removed matter after operation, the width of the clip body 10 is generally 0.3 mm or more and 4 mm or less and the longitudinal length of the clip body 10 in a closed state is generally 5 mm or more and 12 mm or less.

The base end parts A are directly or indirectly connected with the line member (not shown) for moving the clip body 10 in the proximal direction, and are disposed at the most proximal side.

The width of the base end parts A is not limited, however, the maximum width of the base end parts A is preferably smaller than the inner diameter of the tightening member 20. This facilitates the moving of the tightening member 20 in the longitudinal direction without being caught on the base end parts A when the clip body 10 is inserted into the inner space of the tightening member 20. Alternatively, the width of the base end parts A proximal to the tightening member 20 on the base end parts A may be larger than the inner diameter of the tightening member 20. This prevents the tightening member 20 from dropping off from the clip body 10 in the proximal direction.

When the tightening member 20 is composed of an elastic member or a deformable coil-shaped ring member, the maximum width of the base end parts A may be larger than the inner diameter of the tightening member 20. The tightening member 20 being deformed can go over the sections having the maximum width of the base end parts A to move in the proximal direction.

The width of the base end parts A is preferably constant in the longitudinal direction of the base end parts A. This facilitates the moving of the tightening member 20 in the longitudinal direction without being caught on the base end parts A when the clip body 10 is inserted into the inner space of the tightening member 20.

As shown in FIGS. 1 and 2, the two holding members 11 and 12 are preferably joined to each other at the proximal ends of the base end parts A. The clip 1 like this is suitably used for a clip device in which the line member for longitudinally moving the clip body 10 is directly joined to the base end parts A of the holding members 11 and 12 or indirectly joined to the clip body 10 via a joining member to engage with the base end parts A of the holding members 11 and 12 for joining the line member to the clip body 10.

In FIGS. 1 and 2, the two holding members 11 and 12 are integrally formed at the base end parts A by bending one band-shaped member, however, the two holding members 11 and 12 may be joined to each other in other ways, for example, by mechanical fastening such as screwing and caulking, welding, or bonding.

With reference to FIG. 4, an embodiment having the proximal ends of the base end parts A different in shape from those of the clip body 10 shown in FIGS. 1 and 2 will now be described. FIG. 4 is a side view of the clip body of the present invention. As shown in FIG. 4, the two holding members 11 and 12 are preferably joined to each other at the distal end parts of the base end parts A but not at the proximal ends of the base end parts A. The joining position in the base end parts A may appropriately be determined. The clip 1 like this is suitably used for a clip device in which the line member for longitudinally moving the clip body 10 is indirectly connected with the clip body 10 via a joining member (not shown) to engage with the proximal end parts of the base end parts A of the clip body.

As shown in FIG. 4, engaging claws J (11J, 12J) may be provided, facing each other, at the proximal ends of the base end parts A where the two holding members 11 and 12 are not joined to each other. The size and angle of the engaging claws J may appropriately be determined in view of a joining member (not shown) to engage with. The length JL of the engaging claws 11J and 12J is preferably equal to or less than half the maximum distance ADmax between the base end parts A of the two holding members 11 and 12 facing each other, and may be one quarter or more and three quarters or less of the maximum distance ADmax. The angles θ between the engaging claws J and the respective base end parts A of the holding members 11 and 12 is preferably acute angles. The two engaging claws 11J and 12J may be in contact with each other (not shown).

To keep a constant distance between the base end parts A of the holding members 11 and 12 at the proximal sides to facilitate an engaging operation of the clip body 10 with the joining member, a reinforcing member 15 may be provided for joining the base end part 11A of the holding member 11 to the base end part 12A of the holding member 12 as shown in FIG. 4. The reinforcing member is preferably provided at the distal sides of the base end parts A not to impede the engagement of the clip body 10 with the joining member. The reinforcing member 15 is preferably composed of a material similar to the materials for the holding members 11 and 12, and the thickness of the reinforcing member 15 may be determined as the thickness of the holding members 11 and 12 is. The reinforcing member 15 may be mounted by mechanical fastening such as screwing and caulking, welding, or bonding.

The curved parts B are curved inward in the radial direction of the tightening member 20. As shown in FIGS. 1 and 2, the curved parts B of the holding members 11 and 12 are curved inward in the closing direction of the clip body 10. The curved parts B facilitate the bending of the holding members 11 and 12 and the outward spreading of the distal sides of the clip body 10 in the radial direction to adjust a holding position with respect to a target site.

To achieve an appropriate degree of opening and closing of the clip 1, the curvature radius of the curved parts B is preferably 20 mm or more, more preferably 25 mm or more, and much more preferably 30 mm or more, however, is preferably 80 mm or less, more preferably 75 mm or less, and much more preferably 70 mm or less. The curvature radius of the curved parts B is a value under the condition that the tightening member 20 is disposed on the base end parts A of the holding members 11 and 12, or the tightening member 20 is removed from the clip body 10.

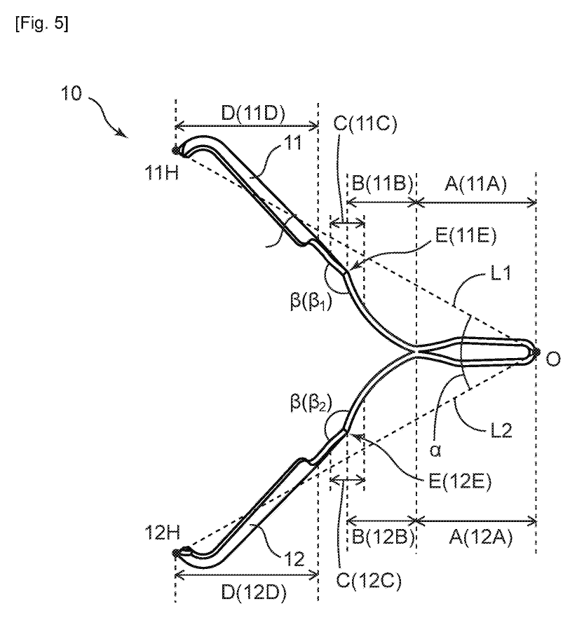

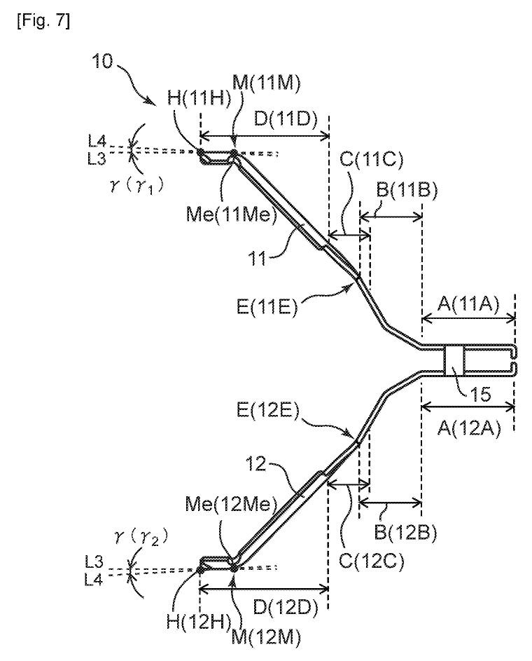

With reference to FIG. 5, a preferable degree of opening of the clip body 10 will now be described. FIG. 5 is a side view of the clip body of the present invention. The clip body 10 is in an open state. The straight line L1 connects the distal end 11H of the holding member 11 and the proximal end O of the clip body 10 while the straight line L2 connects the distal end 12H of the holding member 12 and the proximal end O of the clip body 10. The angle between the straight line L1 and the straight line L2, which is inside in the radial direction of the clip body 10, is defined as an opening angle α. To hold a large target site, the opening angle α is preferably 40° or more, more preferably 50° or more, and much more preferably 60° or more. When there are a plurality of distal ends of the holding members 11 and 12 as shown in FIG. 7 (described below), the distal ends H (11H, 12H) of the holding members 11 and 12, which define the straight lines L1 and L2, mean the distal ends disposed outermost in the radial direction of the clip body 10. When there are a plurality of proximal ends of the clip body 10, the proximal end O of the clip body 10 means the proximal end disposed innermost in the radial direction of the clip body 10.

The wide part C is wider than a minimum width of the curved part B. When the tightening member 20 comes into contact with the wide parts C of the holding members 11 and 12 in the direction perpendicular to the opening-and-closing direction of the clip 1, the wide parts C temporarily stop the tightening member 20 on the holding members 11 and 12.

The wide part C is preferably narrower than a maximum width of the holding part D. This prevents the tightening member 20, which has gone over the bending parts E of the holding members 11 and 12 toward the distal sides of the holding parts D, from dropping off from the clip body 10. Specifically, the maximum width of the holding parts D is preferably 1.25 times or more of the maximum width of the wide parts C, more preferably 1.5 times or more of the maximum width of the wide parts C, and much more preferably 2.0 times or more of the maximum width of the wide parts C.

To temporarily stop the tightening member 20 easily, the maximum width of the wide parts C is preferably 1.5 times or more of the minimum width of the curved parts B, and more preferably 2.0 times or more of the minimum width of the curved parts B. When the wide parts C are wider than the inner diameter of the tightening member 20, the wide parts C may be deformed in the tightening member 20.

To hold a target site easily, the width of the wide parts C is preferably smaller than the inner diameter of the tightening member 20. The width of the wide parts C, however, is preferably at least 70% or more of the inner diameter of the tightening member 20, and more preferably 95% or more of the inner diameter of the tightening member 20. The width of the wide parts C is thus smaller than but close to the inner diameter of the tightening member 20. When the wide parts C are pulled into the tightening member 20, the wide parts C having such a width help the two holding parts 11 and 12 to come close enough to each other to firmly hold a target site. When the two holding members 11 and 12 have claw parts (described below) to engage with each other at the distal ends of the holding parts D, the width of the wide parts C is preferably closer to the inner diameter of the tightening member 20. This enables the tightening member 20 to keep the two holding members 11 and 12 close to each other to engage the claw parts with each other.

The wide parts C are preferably disposed substantially at the same positions of the two holding members 11 and 12 in the longitudinal direction. This enables the tightening member 20 to come into contact with the wide part C of the holding member 11 and the wide part C of the holding member 12 at the same time, which temporarily stops the tightening member 20 easily.

If the wide parts C are largely different in shape from the curved parts B, stress concentration may occur. To avoid the stress concentration, in a plane view of the holding members 11 and 12, the curvature radius of the visible outlines from the distal ends of the sections not including the wide parts C in the curved parts B to the sections having the maximum width of the wide parts C is preferably more than 0.

As shown in FIGS. 1 and 3, the sections including a part of the curved parts B preferably overlap with the sections including a part of the wide parts C. To simplify the production of the clip body 10, the curved parts B, except the sections including a part of the wide parts C, preferably have a constant width in the longitudinal direction.

The curved parts B, except the sections including a part of the wide parts C, preferably have a width smaller than the inner diameter of the tightening member 20. This prevents the tightening member 20 to be caught on the sections not including the wide parts C in the curved parts B when the clip body 10 is inserted into the inner space of the tightening member 20, which facilitates the movement of the tightening member 20 in the longitudinal direction.

The holding parts D directly hold a target site and are disposed at positions distal to the wide parts C.

An inner diameter of the tightening member 20 is preferably shorter than a maximum width of the holding part D. After going over the bending parts E of the holding members 11 and 12 in the distal direction, the tightening member 20 comes into contact with at least the areas having the maximum width of the holding parts D. This prevents the tightening member 20 from moving to the distal sides of the holding parts D and dropping off from the clip body 10.

To hold the target site surely, as shown in FIG. 3, the maximum width of the holding parts D of the holding members 11 and 12 of the clip body 10 is preferably wider than a minimum width of the base end part A. The maximum width of the holding part D is preferably 1.5 times or more of the minimum width of the base end part A, more preferably 2.0 times or more of the minimum width of the base end part A, and much more preferably 3.0 times or more of the minimum width of the base end part A.

To enhance a rigidity of the holding parts D of the holding members 11 and 12, at least a part of the holding part D is preferably formed in an arc shape that is bent outward in the radial direction in a cross-section perpendicular to a longitudinal direction of the holding part D. For example, at least a part of the holding part D is preferably formed in a half-cylindrical shape.

The curvature radius of the areas of the holding parts D having an arc-shaped cross section perpendicular to the longitudinal direction may be determined in view of the inner shape of the inner tubular body of the clip device, or the shape of the forceps channel.

A claw part G is preferably formed at a distal end of the holding part D. The claw parts G cut into a target site when the holding parts D hold the target site, which ensures the holding of the target site. To enable the claw parts G to cut into a target site easily and enhance the stability in a holding operation, the claw parts G may be in the form of teeth. The teeth may be formed so that the claw part 11G of the holding member 11 engages with the claw part 12G of the holding member 12 under the condition that the clip 1 is in a closed state. In FIGS. 1 to 4, the claw parts G have triangle teeth, however, each tooth may have a polygonal shape such as a rectangular shape and a trapezoidal shape.

A curved-surface is preferably formed at the distal end of the holding part D. Specifically, the distal ends of the holding parts D are preferably curved outward in the radial direction. The curves at the distal ends of the holding parts D enable the claw parts G to come into contact with a target site in the direction perpendicular to the longitudinal direction, so that the claw parts G can cut into the target site more easily. In a side view of the clip body 10 in an open state, the holding parts of the clip body 10 preferably have maximum parts M. In the clip body 10 in an open state, the claw parts G are preferably disposed at positions distal to the maximum parts M. The holding parts D may have curves at the angle described below. The radially outermost points of the maximum parts M are defined as maximum points Me. As shown in FIG. 2, the straight lines on the respective maximum points Me and parallel to the longitudinal direction of the clip body 10 in an open state are defined as straight lines L3. The straight lines connecting the distal ends H (11H, 12H) of the holding members 11 and 12 of the clip body 10 in an open state and the respective maximum points Me (11Me, 12Me) of the holding members 11 and 12 are defined as straight lines L4. The angles γ (γ₁, γ₂) between the straight lines L3 and the respective straight lines L4 on the holding members is preferably 20° or more and 50° or less, especially for suturing a target site such as a lesion. This enables the claw parts G to further cut into the target site when the clip 1 is closed. In FIG. 2, both the angles γ₁, γ₂ are at approximately 30°. To pinch a blood vessel for stanching, the angles γ (γ₁, γ₂) between the straight lines L3 and the respective straight lines L4 is preferably −10° or more and less than 15°. This enables the claw parts G to be in contact with a mucous membrane at the right angle (90°) to further cut into a target site when the clip is closed. When there are a plurality of distal ends of the holding members as shown in FIG. 7, the distal ends H (11H, 12H) of the holding members 11 and 12, which define the straight lines L4, mean the distal ends disposed outermost in the radial direction of the clip body 10. In FIG. 7, both the angles γ₁, γ₂ are at approximately −3.5°. The absolute value determined by subtracting the angle γ (γ₂) of the holding member 12 from the angle γ (γ₁) of the holding member 11 may be 0° or more and 5° or less, for example.

The bending parts E are bent outward in the radial direction of the tightening member 20 in the sections including the wide parts C of the holding members 11 and 12. In the present invention, the holding members 11 and 12 of the clip body 10 include the bending parts E. When the tightening member 20, which moves from the proximal side to the distal side, goes over the bending parts E, the tightening member 20 comes into contact with the holding members 11 and 12. At this time, the inner surface of the tightening member 20 applies a pressing force to the holding members 11 and 12 in the direction for closing the clip 1. In the clip 1 of the present invention, as the tightening member 20 moves in an area proximal to the bending parts E of the wide parts C of the holding members 11 and 12 in the longitudinal direction, the tightening member 20 gradually closes the clip 1 in cooperation with the wide parts C to adjust a holding position and a closing angle.

The bending parts E can be formed by bending the holding members 11 and 12 made of a band-shaped member at a desired bending angle (described in detail below) in the simplest way. Alternatively, the bending parts E may be formed by separately preparing members to be disposed proximal to the bending parts E and members to be disposed distal to the bending parts E and joining these two types of members at a desired bending angle by laser welding, for example. Description will be made on the bending angle at which the holding members 11 and 12 are bent to form the bending parts E.

With reference to FIG. 5, the bending angle of the bending parts E will now be described. As shown in FIG. 5, in a side view of the clip body 10, the bending angles β between the curved parts B proximal to the bending parts E and the respective wide parts C distal to the bending parts E, which are inside in the radial direction, are preferably obtuse angles. This ensures a wide view of the operator near the holding parts D to facilitate the holding of a target site, and also ensures the closing of the clip 1 by preventing the tightening member 20 from moving back in the proximal direction after the contact of the tightening member 20 with the bending parts E.

To ensure a wide view of the operator near the holding parts D to facilitate the holding of a target site, the bending angles β are preferably 120° or more, more preferably 125° or more, and much more preferably 130° or more. To ensure the closing of the clip by preventing the tightening member 20 from moving back in the proximal direction after the contact of the tightening member 20 with the bending parts E, the bending angles β are preferably 175° or less, more preferably 170° or less, and much more preferably 165° or less.

As shown in FIG. 5, the absolute value determined by subtracting the bending angle β (β₂) of the holding member 12 from the bending angle β (β₁) of the holding member 11 is preferably 0° or more and 5° or less. This enables the holding member 11 to timely come close to the holding member 12.

The wide part C has a distal section, a proximal section and a central section between the distal section and the proximal section, that are formed by equally dividing the wide part C into three sections in the longitudinal direction of the clip body 10, and the bending part E is preferably formed at the distal section or the central section of the wide part C. This lengthens the section where the clip 1 is gradually closed while adjusting a holding position and a closing angle. To further lengthen the section for adjusting a holding position or a closing angle, the bending parts E are preferably formed in the distal sections of the wide parts C.

It is preferred that an inner diameter of the tightening member 20 is shorter than a maximum width of the holding part D and a length from a proximal end of the holding part D to the bending part E is shorter than the length in the longitudinal direction of the tightening member 20. When the tightening member 20 is in contact with the proximal end of the holding part D, the clip body 10 is closed in the state where the tightening member 20 is in contact with the bending part E, so that the target site is surely held.

As shown in FIGS. 1 and 3, narrow parts F (11F, 12F) that is narrower than a width of the wide part C are preferably formed between the holding part D and the wide part C. This enables the holding of a target site with a smaller power. This is because the narrow part shortens the section to be in contact with the tightening member in the longitudinal direction of the clip body after the distal end of the tightening member goes over the wide parts with the bending parts to come close to the holding parts.

When the length of the tightening member 20 is shorter than the length of the narrow parts F in the longitudinal direction of the clip 1, the tightening member 20, which has gone over the bending parts E of the holding members 11 and 12 in the distal direction, tends to stay in the sections including the narrow parts F. This prevents the tightening member 20 from moving in the longitudinal direction and ensures the closing state of the clip 1. The maximum width of the narrow parts F may be equal to or shorter than the minimum width of the curved parts B or the minimum width of the base end parts A, for example.

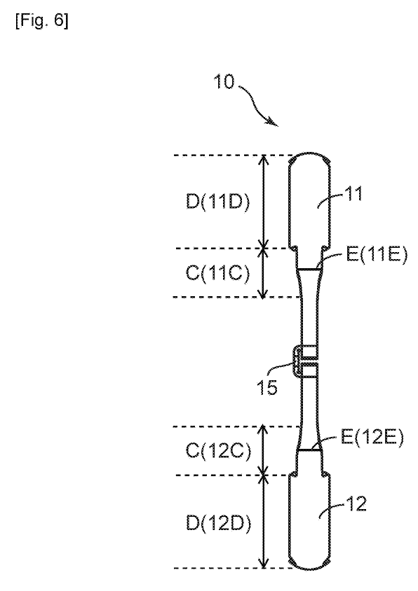

FIGS. 6 and 7 are a rear view and a side view illustrating the clip body according to still another embodiment of the present invention, respectively. As shown in FIG. 6, the narrow parts may be omitted. No difference or a small difference between the width of the narrow parts and the width of the wide parts C increases the strength of the holding parts D of the clip body 10. This enables the holding of a harder mucous membrane.

As shown in FIGS. 1 to 3 and FIG. 5, when the two holding members 11 and 12 are integrally formed at the base end parts A of the clip 1, the two holding members 11 and 12 are preferably in contact with each other at the proximal end parts of the curved parts B. The proximal end part of the curved part 11B of the holding member 11 is in contact with the proximal end part of the curved part 12B of the holding member 12. This enables the adjustment of the degree of opening of the clip body 10 at the distal end and keeps the degree of opening while in use. Compared to the clip having the holding member 11 and the holding member 12 separated from each other at the proximal end parts of the curved parts B, the clip of this type can spread wide at the distal ends of the holding parts D, which facilitates the holding of a target site not only at the distal sides of the holding parts D but also in the entire areas of the holding parts D. In FIGS. 4 and 7, the two holding members 11 and 12 are joined to each other near the curved parts B via the reinforcing member 15. The reinforcing member 15 prevents the distal ends of the holding parts D from spreading too wide. The two holding members 11 and 12 thus may be in contact with each other at the proximal end parts of the curved parts B as shown in FIG. 4. To enable the distal ends of the holding parts D to spread wide, the two holding members 11 and 12 are preferably separated from each other at the proximal end parts of the curved parts B as shown in FIG. 7.

As shown in FIG. 4, the reinforcing member 15 may be disposed at the central positions of the holding members 11 and 12 in the width direction. Alternatively, to facilitate the joint of the holding members and increase the production of clips, the reinforcing member 15 may be disposed on one side of the holding members 11 and 12 in the width direction as shown in FIGS. 6 and 7. To strengthen the joint of the holding members 11 and 12, the reinforcing member 15 may be disposed on both sides of the holding members in the width direction (not shown). The central positions of the holding members in the width direction correspond to the central areas of the base end parts A of the holding members when each base end part A is equally divided into three areas in the width direction.

To secure the length of the holding parts D in the longitudinal direction, the proximal end of the curved part 11B of the holding member 11 is preferably in contact with the proximal end of the curved part 12B of the holding member 12.

To make the holding members 11 and 12 easily deformable to facilitate the holding of a target site, the holding members 11 and 12, especially the sections including the curved parts B, the wide parts C, and the holding parts D may have an opening (not shown). More preferably, the holding parts D of the holding members 11 and 12 have an opening. The shape of the opening(s) is not limited, however, the direction of the major axis of the opening may be parallel to the longitudinal direction of the clip body 10, for example.

The size of the opening is not limited, however, the length of the opening may be 30% or more and 80% or less of the maximal longitudinal length of the holding parts D and the maximal width of the opening may be 10% or more and 80% or less of the maximal width of the holding parts D, for example.

To enhance the strength of the holding members 11 and 12, the holding members 11 and 12 may be embossed to have ridges or grooves (not shown). The holding members 11 and 12 preferably have ridges or grooves at least in the holding parts D, and more preferably in the holding parts D and the curved parts B.

The ridges or grooves may be formed integrally with the holding members 11 and 12 or as separate parts to be jointed with the holding members 11 and 12. When the ridges or grooves are formed integrally, the ridges on one main surface of the holding members 11 and 12 function as the grooves in the other main surface of the holding members 11 and 12. The ridges or grooves are preferably formed along the longitudinal direction of the holding members 11 and 12. The size of the ridges or grooves may be determined as the size of the above openings is.

This application claims the benefit of the priority date of Japanese patent application No. 2015-209355 filed on Oct. 23, 2015. All of the contents of the Japanese patent application No. 2015-209355 filed on Oct. 23, 2015, are incorporated by reference herein.

### DESCRIPTION OF REFERENCE SIGNS

1: a medical clip
10: a clip body
11, 12: a holding member
A, 11A, 12A: a base end part
B, 11B, 12B: a curved part
C, 11C, 12C: a wide part
D, 11D, 12D: a holding part
E, 11E, 12E: a bending part
F, 11F, 12F: a narrow part
G, 11G, 12G: a claw part
J, 11J, 12J: an engaging claw
15: a reinforcing member
20: a tightening member

1. A medical clip comprising:
a ring-shaped tightening member; and
a clip body including two holding members, wherein
each of the two holding members has a base end part, a curved part that is bent inward in a radial direction of the tightening member, a wide part that is wider than a minimum width of the curved part and a holding part for holding a target site in this order from a proximal side of the clip body,
the two holding members are located so as to face each other and are coupled to each other at the base end part,
at least a part of the clip body is located inside the tightening member, and
each of the two holding members has a bending part that is bent outward in the radial direction of the tightening member over the entire width of the holding member at a section of the wide part of the holding member.

2. The medical clip according to claim 1, wherein
the wide part is narrower than a maximum width of the holding part.

3. The medical clip according to claim 1, wherein
each of the two holding members has a narrow part that is narrower than a width of the wide part between the holding part and the wide part in the holding member.

4. The medical clip according to claim 1, wherein
an inner diameter of the tightening member is shorter than a maximum width of the holding part.

5. The medical clip according to claim 1, wherein
the wide part has a distal section, a proximal section and a central section between the distal section and the proximal section, so that the wide part is equally divided into three sections in a longitudinal direction of the clip body, and
the bending part is located at the distal section or the central section of the wide part.

6. The medical clip according to claim 1, wherein
at least a part of the holding part has an arc shape that is bent outward in the radial direction of the tightening member in a cross-section perpendicular to a longitudinal direction of the holding part.

7. The medical clip according to claim 1, wherein
a claw part is located at a distal end of the holding part.

8. The medical clip according to claim 7, wherein
the holding part includes a curved-surface at the distal end of the holding part.

9. The medical clip according to claim 1, wherein
the two holding members are in contact with each other at a proximal end part of the curved part.

10. The medical clip according to claim 1, wherein
the two holding members are integrally formed at the base end part.

11. The medical clip according to claim 1, wherein
the two holding members are not connected to each other at a proximal end of the base end part.

12. The medical clip according to claim 1, wherein the bending part at the wide part of each holding member outwardly is bent so that the clip is closed and the tightening member is prevented from moving in a reverse direction after the tightening member contacts with the bending part.
